(19) United States
(12) Patent Application Publication
(10) Pub. No.: US 2006/0206139 A1
(43) Pub. Date: Sep. 14, 2006

(54) Vascular occlusion device

(76) Inventor: Kurt J. Tekulve, Ellettsville, IN (US)

Correspondence Address: BRINKS HOFER GILSON & LIONE, P.O. BOX 10395, CHICAGO, IL, 60610, US

(21) Appl. No.: 11/334,930

(22) Filed: Jan. 19, 2006

Related U.S. Application Data
(60) Provisional application No. 60/645,375, filed on Jan. 19, 2005.

(51) Int. Cl. A61M 29/00

(52) U.S. Cl. 606/200

(57) ABSTRACT

A vascular occlusion device is provided having bioremodelable materials for occlusion of vessels. The vascular occlusion device may carry an expandable occlusion bag having an internal cavity filled with a filler member, where at least one of the expandable occlusion bag and the filler member contains bioremodelable material. Use of bioremodelable material may provide stable maintenance of the occlusion device in a vessel site so that its migration into the parent vessel is prevented. In particular, the occlusion device may be absorbed into the body and eventually replaced by the individual's own tissue.

This application claims the benefit of priority under 35 U.S.C. § 119(e) to U.S. Provisional Application No. 60/645,375, filed Jan. 19, 2005, which is hereby incorporated by reference in its entirety.

### BACKGROUND

The present invention relates to a vascular occlusion device having bioremodelable collagen-based matrix structures that may be used in situations where occlusion of vessels is desired, including treatment of aneurysms, vascular malformations, arterial fistulas and other vascular disorders.

Aneurysms are the result of a weak area in a vessel wall, resulting in bulging in the weak area at a particular site in the vessel wall. Untreated aneurysms stand the risk of rupturing, which can lead to death.

Conventional endovascular treatment of aneurysms include embolization procedures in which an aneurysm is packed with material preventing the flow of arterial blood therein. Materials used for aneurysm embolization may include platinum coils, such as the FDA approved Gugliemi Detachable Coil. However, this platinum coil is relatively soft and does not provide a complete packing of the aneurysm lumen. It is not uncommon for the aneurysm to re-canalize, enlarge and even rupture. In wider neck aneurysms, embolization coils have been found to migrate back to the parent vessel, which may result in occlusion of the parent vessel. Migration of embolization coils through the blood into other areas can be potentially dangerous.

Embolizing coils also are used in other medical situations where vascular occlusion is desired. Regardless of the situation, a deployment device is typically used to introduce the coils, one by one, usually by way of a catheter, into a desired occlusion site

An alternative to embolizing coils for vascular occlusion involves the use of synthetic, space filling hydrogel agents or particulate materials, including Gelfoam™, Ivalon™, and Oxycel™. These methods similarly suffer a risk of the agents or particulate materials dislodging or causing inappropriate embolization elsewhere or of rupturing in the vessel areas where they were placed. Moreover, the use of synthetic embolization agents may contribute to thrombus formation, immune responses leading to rejection, and incomplete occlusion of the vessel.

Tissue implants having collagen-based materials have been manufactured and disclosed in the literature. Collagen-based materials are desirable in view of their biocompatibility, resorbability and bioremodelable properties. Cohesive films of high tensile strength have been manufactured using collagen molecules or collagen-based materials. Aldehydes, however, have been generally utilized to cross-link the collagen molecules to produce films having high tensile strengths. With these types of materials, the aldehydes may leech out of the film, e.g. upon hydrolysis. Because such residues are cytotoxic, the films have disadvantages where used as tissue implants.

Other techniques have been developed to produce collagen-based tissue implants while supposedly avoiding the problems associated with aldehyde cross-linked collagen molecules. One such technique is illustrated in U.S. Pat. No. 5,141,747 where the collagen molecules are cross-linked or coupled at their lysine epsilon amino groups followed by denaturing the coupled, and preferably modified, collagen molecules. However, such biomaterials are not bioremodelable or capable of stable absorption into bodily tissues.

### SUMMARY

In one approach, a vascular occlusion device is provided having an expandable occlusion bag and a filler member where at least one of the expandable bag and the filler member includes bioremodelable material and where the filler member is transferrable to the internal cavity through the opening of the expandable bag, such that it can fill and expand the bag to facilitate occlusion in a body vessel. Exemplary embodiments include naturally-derived collagenous extracellular matrix materials (ECMs), such as submucosal materials for use in the bag, the filler member, or both. Marker materials may be added to the filler and/or bag materials to render the device radiopaque or MRI compatible.

The bioremodellable materials may be degraded and replaced by endogenous tissues upon implantation in a host. This results in better anchoring of the device or even permanent replacement of the device by the patient's endogenous tissues to stably maintain the vessel occlusion and/or prevent migration of the occlusion device back into the parent vessel or elsewhere.

In another aspect, a method for occluding a vessel in a patient is provided in which an expandable occlusion bag is positioned in a vessel of a patient, a filler member is transferred into the internal cavity of the occlusion bag and the occlusion bag is expanded with the filler member so that the occlusion bag expands, thereby occluding the vessel.

In a further aspect, a method for stably occluding a vessel with endogenous tissue from a patient is provided in which an expandable occlusion bag and a filler member, each consisting essentially of a bioremodelable or ECM material, is positioned in a vessel so that the filler member enclosed occlusion bag is allowed to undergo remodeling such that the bioremodelable or ECM is degraded and replaced by endogenous tissue.

In another aspect, a vascular occlusion assembly is provided having a catheter assembly joined to a vascular occlusion device. The vascular occlusion assembly contains a vascular occlusion device having an expandable occlusion bag capable of being filled with a filler member to occlude a vessel in a body, and is further joined to catheter assembly having a positioning catheter in which the distal end of the positioning catheter is joined to an opening in the expandable occlusion bag, catheter having a means for: positioning the occlusion bag in a vessel of a patient; aiding in the transfer of the filler member into the occlusion bag expanding its shape to occlude the vessel; and providing a disengagement function to facilitate the release of the filled expandable occlusion bag into the vessel of the patient.

In a further aspect, a method for occluding a vessel in a patient in which a catheter assembly is provided having a means for delivering a vascular occlusion device into a vessel of a patient, where the catheter assembly positions the expandable occlusion bag in a vessel of a patient; aiding in the transfer of the filler member into the occlusion bag and the resultant expansion in the shape of the occlusion bag to occlude the vessel; and disengaging the occlusion bag from the catheter assembly.

Other features, methods and advantages of the invention will be, or will become, apparent to one with skill in the art upon examination of the following figures and detailed description. It is intended that all such additional systems, methods, features and advantages are included within this description, are within the scope of the invention, and are protected by the following claims.

### BRIEF DESCRIPTION OF THE DRAWINGS

FIG. 1 depicts a partially sectioned, longitudinal view of an occlusion device positioned in a blood vessel having an expandable occlusion bag in an expanded shape containing a filler member connected to a positioning catheter.

FIG. 2 depicts a partially sectioned, longitudinal view of the device of FIG. 1 with the expandable occlusion bag expanded and released from the positioning catheter.

FIG. 3 depicts an enlarged view of distal end of an occlusion device embodiment having a coil as the filler member.

FIG. 4 depicts a partially sectioned, longitudinal view of a representative vascular occlusion assembly of the present invention.

FIG. 5 depicts a longitudinal view of the releasable attachment of the handle from the filler member coil in FIG. 3.

### DETAILED DESCRIPTION

In order to provide a clear and consistent understanding of the specification and claims, the following definitions are provided.

As used herein, the term “vessel” is defined as including any bodily canal, conduit, duct or passageway, including but not limited to blood vessels, bile ducts, the esophagus, the trachea, the ureter and the urethra.

The term “expanded occlusion bag” refers to an expandable occlusion bag filled with filler member.

The term “bioremodelable” refers to a natural or synthetic material capable of inducing tissue remodeling in a subject or host. A bioremodelable material includes at least one bioactive agent (e.g., growth factor, etc.) capable of inducing tissue remodeling. The ability to induce tissue remodeling may be ascribed to one or more bioactive agents in a bioremodelable material stimulating the infiltration of native cells into an acellular matrix, stimulating new blood vessel formation (capillaries) growing into the matrix to nourish the infiltrating cells (angiogenesis), and/or effecting the degradation and/or replacement of the bioremodelable material by endogenous tissue. The bioremodelable material may include extracellular collagen matrix (ECM) material, including but not limited to submucosal tissue, such as small intestine submucosal (SIS) tissue or it may include other natural tissue source materials, or other natural or synthetic materials, including one or more bioactive substances capable of inducing tissue remodeling.

The terms “angiogenesis and angiogenic” refer to bioremodelable properties defined by formation of capillaries or microvessels from existing vasculature in a process necessary for tissue growth, where the microvessels provide transport of oxygen and nutrients to the developing tissues and remove waste products.

The term “submucosa” refers to a natural collagen-containing tissue structure removed from a variety of sources including the alimentary, respiratory, intestinal, urinary or genital tracts of warm-blooded vertebrates. Submucosal material according to the present invention includes tunica submucosa, but may include additionally adjacent layers, such the lamina muscularis mucosa and the stratum compactum. A submucosal material may be a decellularized or acellular tissue, which means it is devoid of intact viable cells, although some cell components may remain in the tissue following purification from a natural source. Alternative embodiments (e.g., fluidized compositions etc.) include submucosal material expressly derived from a purified submucosal matrix structure. Submucosal materials according to the present disclosure are distinguished from collagen materials in other occlusion devices that do not retain their native submucosal structures or that were not prepared from purified submucosal starting materials first removed from a natural submucosal tissue source.

The term “small intestinal submucosa” (SIS) refers to a particular type of submucosal structure removed from a small intestine source, such as pig.

The term “radiopaque” is defined as a non-toxic material capable of being monitored or detected during injection into a mammalian subject by, for example, radiography or fluoroscopy. The radiopaque material may be either water soluble or water insoluble. Examples of water soluble radiopaque materials include metrizamide, iopamidol, iothalamate sodium, iodomide sodium, and meglumine. Examples of water insoluble radiopaque materials include tantalum, tantalum oxide, and barium sulfate, which are commercially available in the proper form for in vivo use. Other water insoluble radiopaque materials include, but are not limited to, gold, tungsten, stainless steel, and platinum.

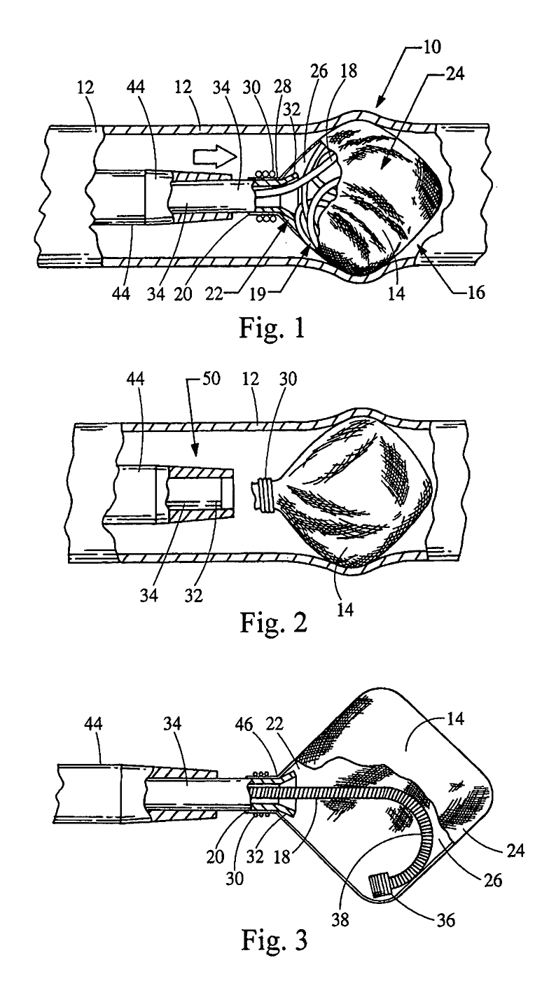

FIG. 1 depicts a partially sectioned, longitudinal view of an occlusion device 10 positioned in a blood vessel 12 having an expandable occlusion bag 14 in an expanded shape 16 containing a filler member 18. When the filler member 18 is convoluted 19 and positioned in the expandable occlusion bag 14, the bag can assume an expanded shape 16 so as to occlude a vessel in a body.

The expandable occlusion bag 14 may be diamond shaped, for example, as shown in FIGS. 1-4, or it may accommodate other shapes suitable for expanding the occlusion bag 14 to occlude a vessel, including circular, spherical, cylindrical, oval and the like. Except for the opening 20 in the expandable occlusion bag 14, which permits transfer of the filler member 18 into the internal cavity 22, the expandable occlusion bag 14 may be partially, substantially, or completely closed, depending on the extent to which the filler member 18 is capable of expanding the internal cavity 22 and of being retained therein. Preferably, the size of the occlusion bag 14 ranges from 3 to 9 mm wide. However, the size of the bag 14 may be larger or smaller depending on the size of the vessel 12 to be occluded.

In a preferred embodiment, the occlusion bag 14 may include or consist essentially of a bioremodelable material. An occlusion device having e.g., natural, bioremodelable materials can better facilitate partial or complete resorption of the occlusion device in a patient's body. Occlusion devices having natural, bioremodelable materials, including ECM materials, are thought to be less thrombogenic and less immunogenic compared to occlusion devices made from synthetic materials. Use of bioremodelable materials may allow for greater occlusion of the vessel and may provide the further benefit of being stably resorbed into the body through replacement by an individual's own tissue.

When used in an occlusion device, the bioremodelable material can undergo remodeling, which may include: (1) stimulation in the infiltration of native cells into an acellular matrix; (2) stimulation of new blood vessel formation (capillaries) growing into the matrix to nourish the infiltrating cells (angiogenesis); and/or (3) effecting the degradation and/or replacement of the bioremodelable material by endogenous tissue upon implantation into a host.

Bioremodelable materials have been used successfully in vascular grafts, urinary bladder and hernia repair, replacement and repair of tendons and ligaments, and dermal grafts. When used in such applications, the graft constructs appear not only to serve as a matrix for the regrowth of the tissues replaced by the graft constructs, but also to promote or induce such regrowth of endogenous tissue. Common events in the remodeling process include widespread, rapid neovascularization, proliferation of granulation mesenchymal cells, biodegradation/resorption of implanted intestinal submucosal tissue material, and lack of immune rejection. The occlusion device may be positioned at a vessel site where it is ultimately replaced by endogenous tissue. Bioremodelable materials for use in the present invention may possess one or more angiogenic properties. Angiogenesis represents a crucial step in tissue formation in response to biomaterial implantation, especially necessary for implants that are designed to foster tissue growth.

Angiogenesis is a complex process that depends on many mechanisms occurring in an organized manner (P. Carmeliet, Mechanisms of angiogenesis and arteriogenesis, Nat Med 6 (2000), no. 4, 389-395). Due to the complexity necessary for proper angiogenesis, biomaterial interaction with the host environment can have a dramatic effect on the quality and quantity of the angiogenic activity. Methods for measuring in vivo angiogenesis in response to biomaterial implantation have recently been developed. One such method uses a mouse subcutaneous implant model to determine the angiogenic potential (Heeschen, C. et al., Nat Med vol. 7, no. 7, pp. 833-839, 2001). When combined with a fluorescence microangiography technique (Johnson, C. et al., Circ Res., vol. 94, no. 2, pp. 262-268, 2004), this model can give quantitative and qualitative measures of angiogenesis into biomaterials.

Bioremodelable materials for use with the present invention may include naturally-derived collagenous ECM materials isolated from suitable animal or human tissue sources. As used herein, it is within the definition of a “naturally-derived ECM” to clean, delaminate, and/or comminute the ECM, or to cross-link the collagen or other components within the ECM. It is also within the definition of naturally occurring ECM to fully or partially remove one or more components or subcomponents of the naturally occurring matrix.

Bioremodelable materials, including ECM materials and others, possess biotropic properties capable of inducing tissue remodeling. Suitable ECM materials include, for example, submucosal (including for example small intestinal submucosa (SIS), stomach submucosa, urinary bladder submucosa, or uterine submucosa, each of these isolated from juvenile or adult animals), renal capsule membrane, dermal collagen, amnion, dura mater, pericardium, serosa, peritoneum or basement membrane layers or materials, including liver basement membrane or epithelial basement membrane materials. These materials may be isolated and used as intact natural sheet forms, or reconstituted collagen layers including collagen derived from these materials, or in a form of foam or a sponge, and/or other collagenous materials may be used. For additional information as to submucosa materials useful in the present invention, and their isolation and treatment, reference can be made to U.S. Pat. Nos. 4,902,508, 5,554,389, 5,733.337, 5.993,844, 6,206,931, 6,099,567, and 6,331,319. Renal capsule membrane can also be obtained from warm-blooded vertebrates, as described more particularly in International Patent Application serial No. PCT/US02/20499, published as WO 03002165. Commercially available ECM materials capable of remodeling to the qualities of its host when implanted in human soft tissues include porcine SIS material (Surgisis® line of SIS materials, Cook Biotech Inc., West Lafayette, Ind.) and bovine pericardium (Peri-Strips®), Synovis Surgical Innovations, St. Paul, Minn.).

The following U.S. patents, hereby incorporated by reference, disclose the use of ECMs for the regeneration and/or repair of various tissues: U.S. Pat. Nos. 6,379,710; 6,187,039; 6,176,880; 6,126,686; 6,099,567; 6,096,347; 5,997,575; 5,993,844; 5,968,096; 5,955,110; 5,922,028; 5,885,619; 5,788,625; 5,762,966; 5,755,791; 5,753,267; 5,733,337; 5,711,969; 5,645,860; 5,641,518; 5,554,389; 5,516,533; 5,460,962; 5,445,833; 5,372,821; 5,352,463; 5,281,422; and 5,275,826.

Preferred ECM materials contain residual bioactive proteins or other ECM components derived from the tissue source of the materials. For example, they may contain Fibroblast Growth Factor 2 (basic FGF), vascular endothelial growth factor (VEGF), and Transforming Growth Factor-beta (TFG-beta). It is also expected that ECM base materials of the invention may contain additional bioactive components including, for example, one or more of glycosaminoglycans, glycoproteins, proteoglycans, and/or growth factors.

Submucosal materials, including SIS materials, represent preferred examples of ECM materials for use with the present invention. The ECM materials may include residual bioactive proteins or other ECM components derived from the tissue source of the materials. The ECM materials may include (among others) fibroblast growth factor 2 (FGF-2), vascular endothelial growth factor (VEGF), transforming growth factor-beta (TGF-beta). It is also expected that ECM base materials of the invention may contain additional bioactive components including, for example, one or more highly conserved collagens, growth factors, glycoproteins, proteoglycans, glycosaminoglycans, other growth factors, and other biological materials such as heparin, heparin sulfate, hyaluronic acid, fibronectin and the like. Thus, generally speaking, submucosal or other ECM materials may include a bioactive agent capable of inducing, directly or indirectly, a bioremodeling response reflected in a change in cell morphology, proliferation, growth, protein and/or gene expression. The bioactive agents in the ECM materials may be contained in their natural configuration and natural concentration.

ECM or submucosal materials may be isolated from warm-blooded vertebrate tissues including the alimentary, respiratory, intestinal, urinary or genital tracts of warm-blooded vertebrates. Preferred submucosal tissues may include intestinal submucosa, stomach submucosa, urinary bladder submucosa, and uterine submucosa. Intestinal submucosal tissue is one preferred starting material, and more particularly intestinal submucosa delaminated from both the tunica muscularis and at least the tunica mucosa of warm-blooded vertebrate intestine.

An exemplary submucosa material is small intestine submucosa (SIS). SIS has been shown to be acellular, strong, and exhibit a sidedness in that it has a differential porosity of its mucosal and serosal sides. Highly purified SIS generally does not trigger any negative immune system responses, generally is free of viral activity, and is known to reduce seepage. A preferred intestinal submucosal tissue source in accordance with the present invention is porcine SIS.

The preparation of intestinal submucosa is described and claimed in U.S. Pat. Nos. 6,206,931 and 6,358,284, the disclosures of which are expressly incorporated by reference in their entirety. Urinary bladder submucosa and its preparation is described in U.S. Pat. No. 5,554,389, the disclosure of which is expressly incorporated herein by reference in its entirety. Stomach submucosa and its preparation is described in U.S. Pat. No. 6,099,567, the disclosure of which is expressly incorporated herein by reference in its entirety.

Preferred SIS material typically includes the tunica submucosa delaminated from both the tunica muscularis and at least the luminal portions of the tunica mucosa. The submucosal tissue may include the tunica submucosa and basilar portions of the tunica mucosa including the lamina muscularis mucosa and the stratum compactum. The preparation of intestinal submucosa is described in U.S. Pat. No. 4,902,508, and the preparation of tela submucosa is described in U.S. Pat. Nos. 6,206,931 and 6,358,284, all of which are incorporated herein by reference. The preparation of submucosa is also described in U.S. Pat. No. 5,733,337; Nature Biotechnology, vol. 17, p. 1083 (November 1999); and WO 98/22158. Also, a method for obtaining a highly pure, delaminated submucosa collagen matrix in a substantially sterile state was previously described in U.S. Pat. Pub. No. 2004/180042, which is incorporated by reference herein.

A preferred purification process involves disinfecting the submucosal tissue source, followed by removal of a purified matrix including the submucosa. It is thought that delaminating the disinfected submucosal tissue from the tunica muscularis and the tunica mucosa minimizes exposure of the submucosa to bacteria and other contaminants following delamination and better preserves the aseptic state and inherent biochemical form of the submucosa to potentiate its beneficial effects. Alternatively, the ECM- or submucosa may be purified a process in which the sterilization step is carried out after delamination as described in U.S. Pat. Nos. 5,993,844 and 6,572,650.

The stripping of the submucosal tissue source is preferably carried out by utilizing a disinfected or sterile casing machine, to produce submucosa, which is substantially sterile and which has been minimally processed. A suitable casing machine is the Model 3-U-400 Stridhs Universal Machine for Hog Casing, commercially available from the AB Stridhs Maskiner, Gotoborg, Sweden. As a result of this process, the measured bioburden levels may be minimal or substantially zero. Other means for delaminating the submucosa source can be employed, including, for example, delaminating by hand.

In this method, a segment of vertebrate intestine, preferably harvested from porcine, ovine or bovine species, may first be subjected to gentle abrasion using a longitudinal wiping motion to remove both the outer layers, identified as the tunica serosa and the tunica muscularis, and the innermost layer, i.e., the luminal portions of the tunica mucosa. The submucosal tissue is rinsed with water or saline, optionally sterilized, and can be stored in a hydrated or dehydrated state. Delamination of the tunica submucosa from both the tunica muscularis and at least the luminal portions of the tunica mucosa and rinsing of the submucosa provide an acellular matrix designated as submucosal tissue. The use and manipulation of such material for the formation of ligament and tendon grafts and the use more generally of such submucosal tissue constructs for inducing growth of endogenous connective tissues is described and claimed in U.S. Pat. No. 5,281,422, disclosure of which is incorporated herein by reference.

Following delamination, submucosa may be sterilized using any conventional sterilization technique including propylene oxide or ethylene oxide treatment and gas plasma sterilization. Sterilization techniques which do not adversely affect the mechanical strength, structure, and biotropic properties of the purified submucosa are preferred. Preferred sterilization techniques also include exposing the graft to ethylene oxide treatment or gas plasma sterilization. Typically, the purified submucosa is subjected to two or more sterilization processes. After the purified submucosa is sterilized, for example by chemical treatment, the matrix structure may be wrapped in a plastic or foil wrap and sterilized again using electron beam or gamma irradiation sterilization techniques.

Preferred submucosa may be characterized by the low contaminant levels set forth in Table 1 below. The contaminant levels in Table 1 may be found individually or in any combination in a given ECM sample. The abbreviations in Table 1 are as follows: CFU/g=colony forming units per gram; PFU/g=plaque forming units per gram; ig/mg=micrograms per milligram; ppm/kg=parts per million per kilogram.

TABLE 1

| | First Preferred | | Second Preferred | | Third Preferred | |
|---|---|---|---|---|---|---|
| | Level | | Level | | Level | |
| ENDOTOXIN | <12 | EU/g | <10 | EU/g | <5 | EU/g |
| BIOBURDEN | <2 | CFU/g | <1 | CFU/g | <0.5 | CFU/g |
| FUNGUS | <2 | CFU/g | <1 | CFU/g | <0.5 | CFU/g |
| NUCLEIC | <10 | ig/mg | <5 | ig/mg | <2 | ig/mg |
| ACID | | | | | | |
| VIRUS | <500 | PFU/g | <50 | PFU/g | <5 | PFU/g |
| PROCESSING | <100,000 | ppm/kg | <1,000 | ppm/kg | <100 | ppm/kg |
| AGENT | | | | | | |

ECM- or submucosa materials may be optimally configured by stretching or by laminating together multiple pieces, layers or strips of submucosal tissue compressed under e.g., dehydrating conditions in accordance with the teachings set forth in U.S. Pat. Nos. 6,206,931 and 6,358,284. As disclosed in the '931 and '284 patents, depending on the manner in which the pieces are overlayed together, multilaminate compositions may be engineered with different isotropic properties.

SIS in its normal sheet form has widely varying differences in its thickness and porosity on any given piece of material. Instead of using the SIS material in its normally occurring sheet form, the SIS may be cut into pieces or can be shredded or ground into small sized bits or particles. These small pieces or bits may then be uniformly sprayed, formed, coated or cast on to one or more parts of the vascular occlusion device, such as the occlusion bag or filler material, or on to a mandrel or mold of the appropriate shape and size for one or more components of the vascular occlusion device. The malleable, hydrated pieces may be cast on or applied like papier mache to a form. After the cast is dried or allowed to harden, the form can be removed. The SIS particles can be sprayed, coated or cast onto one or more components of the occlusion device materials or mandrel with or without a binder material to enhance the physical strength of the resulting structure.

ECM or submucosal tissue of the present invention may be further processed into sheet form, chunks, or alternatively, in fluidized or powdered forms. SIS material may be in a form of a sponge-like or foam-like SIS (lyophilized SIS sponge, such as SURGISIS™ Soft-Tissue Graft (SIS) [Cook Biotech, Inc., West Lafayette, Ind.]) capable of greatly expanding in diameter as it absorbs therapeutic material, or non-sponge material including a sheet of SIS. Fluidized or powdered forms of submucosa may be prepared using the techniques described in U.S. Pat. No. 6,206,931 or U.S. Pat. No. 5,275,826, the disclosure of which is expressly incorporated herein by reference in its entirety.

The viscosity of fluidized submucosa compositions for use in accordance with this invention may be manipulated by controlling the concentration of the submucosa component and the degree of hydration. The viscosity may be adjusted to a range of about 2 to about 300,000 cps at 25.degree. C. Higher viscosity formulations, for example, gels, may be prepared from the submucosa digest solutions by adjusting the pH of such solutions to about 6.0 to about 7.0.

ECM or submucosal materials may be stored in a hydrated or dehydrated state. Lyophilized or air dried submucosa materials may be rehydrated and used in accordance with this invention without significant loss of its biotropic, thromboresistant or mechanical properties.

The bioremodelable materials for use with the present invention are not limited to ECM materials. A bioremodelable material may also include a natural and/or resorbable material including at least one bioactive agent and/or growth factor capable of inducing tissue remodeling. Other commercially available remodelable materials include harvested skin from cadaveric donors (Alloderm®, LifeCell Corp., Branchburg, N.J.).

While naturally derived biomaterials, particularly bioremodelable materials, such as SIS, are generally preferred, synthetic materials, including those into which growth factors or other bioactive agents are added to make them bioremodelable, are also within the scope of the present invention.

An expandable occlusion bag 14 including bioremodelable material may be made in several different ways. For example, the bioremodelablel material may be pressed together in the form of a sturdy pouch assembled by processes including laser welding, bonding, sewing, or through the use of pressure, heat, or the use of adhesive substances, such as glue. Representative processing procedures are disclosed in U.S. Pat. No. 6,358,284.

The expandable occlusion bag 14 may also be made from synthetic materials, and may contain, for example, mated pieces 24 and 26 of untreated rip stop nylon attached together about the circumference thereof by heat sealing or any other suitable attachment method.

The expandable occlusion bag 14 further includes an external opening 20, which communicates with internal cavity 22, and may further include a neck 28 positioned between the external opening 20 of the occlusion bag 14 and the internal cavity 22. The expandable occlusion bag 14 may further include a collar 30 positioned around neck 28 for retaining a flared distal end 32 of a positioning catheter 34 in the internal cavity 22 of the expandable occlusion bag 14. Collar 30 may contain, for example, several turns of 0.004 inch diameter platinum wire wrap, such as commercially available tungsten/platinum alloy #479 wire. Collar 30 may, for example, be attached to neck 28 using commercially available medical grade adhesive or any other suitable means for attachment.

A vascular occlusion device 10 containing an expandable occlusion bag 14 further includes a filler member 18. In a preferred embodiment, the filler member 18 may include or consist essentially of submucosal material. When bioremodelable material is used in the filler member, it may be present in sheet form, chunks or in fluidized or powdered forms. The bioremodelable materials may be further configured by stretching, by laminating together multiple pieces, layers or strips.

In a particularly preferred embodiment, an occlusion device 5 is provided in which both the expandable bag 14 and the filler member 18 include or consist essentially of submucosal material.

A filler member 18 may include synthetic materials in the form of a coil or wire. FIG. 3 depicts a representative embodiment in which the filler member includes a synthetic material in the form of a coil. Embolization coil(s) or wire(s) for use as embolizing agents are widely known in the art. They can also be used as a filler member 18 within the context of the present invention and may be made from various metal or metal alloy materials, including platinum, stainless steel, and nickel-based alloys, such as Inconel™. Representative coils are disclosed in Gianturco U.S. Pat. No. 5,334,210, the disclosure of which is incorporated herein by reference in its entirety.

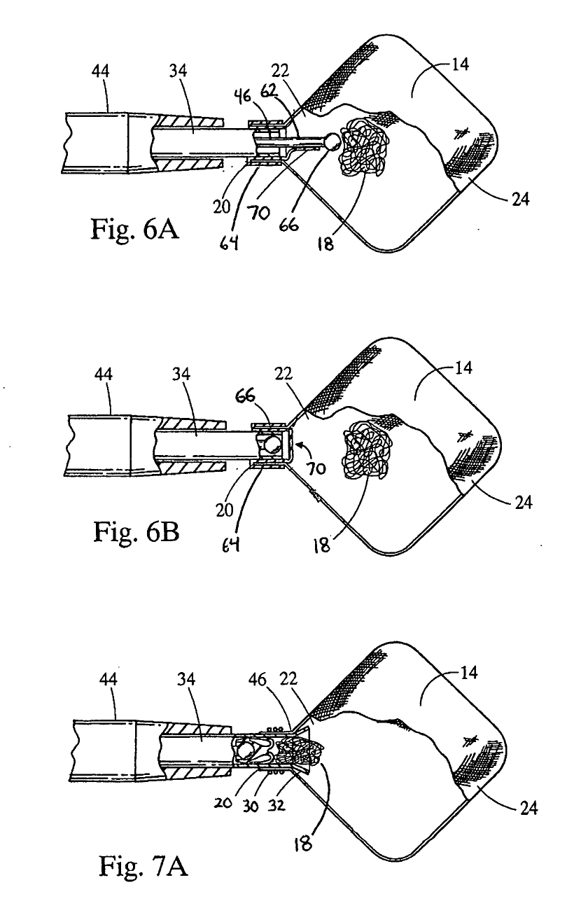

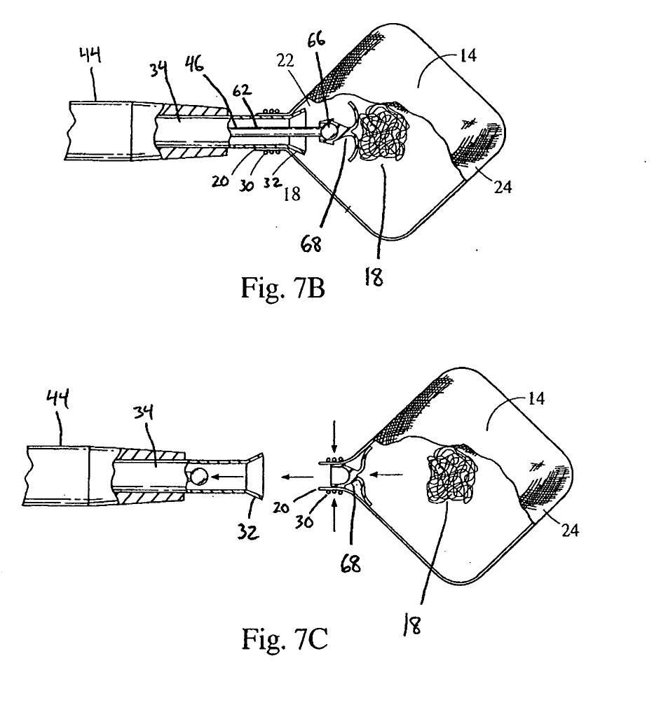

Coils or wires may include, for example, an enlarged distal end segment 36 for preventing protrusion and release of the filler member through the expandable occlusion bag 14 when filler member 18 is positioned therein (FIG. 3). The enlarged cross-sectional dimension of distal end segment 36 may be designed to prevent puncture of the expandable occlusion bag 14. Additionally, the enlarged distal end segment may also be designed to prevent the filler member 18 from being pulled back through the positioning catheter 34. For example, filler member 18 may include, a helical coil with a proximal end 38 as depicted in FIG. 5, and discussed below, and a preformed hook-shaped distal portion 36 to facilitate bending of the member 18 into a convoluted configuration 19 (FIG. 1) during advancement into the cavity of the expandable occlusion bag 14 and to prevent the filler member 18 from being pulled back through the positioning catheter 34. Filler member 18 may include, for example, an appropriate length of 0.025″ diameter stainless steel coil to fill the occlusion bag 14. Enlarged distal end segment 36 may contain, for example, a 0.050″ length of 0.045″ diameter stainless steel coil welded to the distal end of member 18.

A representative forming wire that may be utilized in the present invention is disclosed in Grifka et al., Circulation, 91:1840-1846 (1995). Upon its release into the expandable bag, the forming wire should assume a suitable form or shape expanding the bag to sufficiently occlude a vessel. The forming wire(s) for use in the present invention may include metal or metal alloy materials, including platinum, stainless steel, gold, and nickel-based alloys, such as Nitinol™ and Inconel™. The wire(s) may be heat-set or pre-shaped to assume a desired shape consistent with expansion and occlusion of a vessel upon release of the wire into the inner cavity of the bag.

In a preferred embodiment, the expandable occlusion bag 14 and filler member 18 may both include submucosal materials. The expandable occlusion bag 14 and filler member 18 may further include radiopaque marker materials, such as platinum or tungsten, as described in U.S. 2003/0206860, the disclosure of which is incorporated herein by reference in its entirety. The radiopaque marker materials may be included within any aspect of the vascular occlusion device. For example, the radiopaque marker materials may also be included with the submucosal materials and may be incorporated between one or more layers of submucosal structure material(s). Alternatively, radiopaque marker materials may be introduced in powdered form or any other form suitable for rendering the occlusion device radiopaque.

The radiopaque marker materials may also be included with synthetic materials present in the expandable occlusion bag 14 and/or the filler member 18. For example, radiopaque materials, such as platinum or tungsten, may be used to make the coils or wires in the filler member. Alternatively, the radiopaque materials may be included among the synthetic materials in the coils or wires.

A further aspect of the present invention provides a method for occluding a vessel in a patient in which a catheter assembly 42 is provided having a means for delivering a vascular occlusion device 10 into a vessel 12 of a patient, where the catheter assembly 42 positions the expandable occlusion bag 14 in a vessel 12 of a patient; where the catheter assembly 42 aids in the transfer of the filler member 18 into the occlusion bag 14, thereby expanding the shape of the occlusion bag to occlude the vessel 12; and where the catheter assembly 42 provides a means for disengagement, release, and/or closure of the expanded occlusion bag 14 to prevent release of the filler member 18 into the vessel 12.

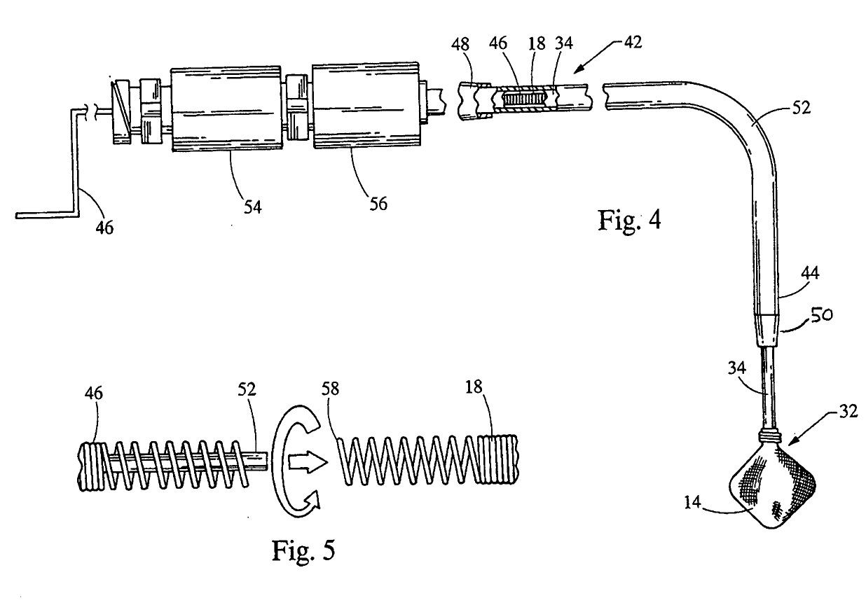

Accordingly, methods for occluding a vessel in a body are provided in which a catheter assembly 42 is used to deliver, position, and release a vascular occlusion device 10 at a vessel site for occlusion, where the expandable occlusion bag 14 is filled with the filler member 18 to occlude the vessel 12 (FIG. 4). Conventional catheters may be used to position and deliver an occlusion bag 14 in accordance with the present invention. Although no limitation is intended, representative catheter assemblies for positioning and delivering an occlusion device 10 of the present invention are disclosed in Gianturco, U.S. Pat. No. 5,334,210; Hall et al., U.S. Pat. No. 5,417,708; Parker, U.S. Pat. No. 5,562,698; Rasmussen, U.S. Pat. No. 5,725,534; and Klint, U.S. Pat. No. 6,458,137, the disclosures of which is incorporated herein by reference in its entirety. While these devices particularly describe the positioning, delivery, and release of an occlusion device in which the filler member is in the form of coil(s) or wire(s), the catheter devices described therein may be readily adapted for releasing a filler material including bioremodelable materials into the expandable bag 14.

In a preferred embodiment, an expandable occlusion bag 14 may be introduced in a vascular occlusion site with the assembly depicted in FIG. 4 using, for example, the well-known Seldinger technique. FIG. 4 depicts a partially sectioned, longitudinal view of an illustrative vascular occlusion assembly 42, including an expandable occlusion bag 14, positioning catheter 34, pusher catheter 44, handle 46, and introducer sheath 48. Accordingly, this assembly provides an expandable bag 14 having a filler member 18 releasably delivered into the bag by actuating a handle 46 associated with a pusher catheter 44 and a positioning catheter 34. A filler member 18 is transferred into the internal cavity of the occlusion bag, thereby expanding its volume and shape to occlude the vessel 12. Actuating the handle 46 releases the filler member 18 and a pusher catheter 44 disengages the bag 14 from the positioning catheter 34.

An expandable occlusion bag 14 of the coaxial vascular occlusion assembly depicted in FIG. 4 may be percutaneously positioned in a vascular site such as blood vessel 12. Filler member 18 may be positioned in internal cavity 22 of the expandable occlusion bag 14 by pushing distally on the handle 46. The handle can be rotated proximally for detaching or releasing the handle 46 from the filler member 18. A beveled distal end 50 of pusher catheter 44 can be advanced from introducer sheath 48 along positioning catheter 34 to engage and release a neck 28 of the expanded occlusion bag 14 from a flared distal end 32 of the positioning catheter 34.

In FIG. 4, the occlusion bag 14 is connected to a positioning catheter 34. The expandable occlusion bag 14 may be positioned about a flared distal end 32 of positioning catheter 34 (FIG. 1, 3). An introducer sheath 48 and positioning and pusher catheters 34, 44, respectively, may be coaxially positioned for introduction and release of the occlusion bag 14 at a desirable site in the vascular system of a patient (FIG. 4). Pusher catheter 44 may include curved distal portion 52 for directional control of the vascular occlusion assembly in the vascular system. Coaxial positioning catheter 34 may similarly include a curved distal portion (not shown). Coaxial catheters 34 and 44 can be fixedly positioned relative to each other with well-known interconnecting proximal hubs 54 and 56, respectively.

Pusher catheter 44 may include, for example, an 86 cm length of 7.5 French, 0.0985″ outside diameter, TEFLON™ radiopaque tubing material. Positioning catheter 34 may include, for example, an 87 cm length of 4 French 0.053″ outside diameter, TEFLON™ radiopaque tubing material.

A beveled distal end 50 of pusher catheter 44 can provide for release of the expandable occlusion bag 14 from a flared distal end 32 of the positioning catheter 34. Pusher catheter 44 is advanced distally over the flared distal end 32 of positioning catheter 34 for flattening or compressing the flare. When the flare is compressed, a filled expandable occlusion bag 14 can be readily released from positioning catheter 34 (FIG. 1-2).

FIG. 5 depicts a representative longitudinal view of the releasable attachment of handle 46 and proximal end 58 of filler member 18, including a screw type connection release mechanism, where the filler member 18 is in the form of a coil. In this case, the handle 46 and filler member 18 are attached by rotating the handle 46 for engaging or attaching the mating coils 46, 58. Prior to introduction into the vascular system of a patient, the vascular occlusion assembly is coaxially positioned with handle 46 and filler member 18 releasably attached at proximal end 58 thereof and positioned in the positioning catheter 34.

Where the filler member 18 includes coils, the filler member 18 may have, for example, a 6 to 8 mm length of coils about proximal end 58 that are spaced from each other approximately 0.010 mm (FIG. 5). The preformed hook-shaped distal portion 36 has, for example, an approximately 2.5 mm J-curve. Handle 64 contains, for example, a 73.5 cm length of 0.025″ diameter stainless steel coil with a 6 to 8 mm length of distal coils that are separated from each other approximately 0.010 mm and positioned around distal end mandril wire 52. The separated handle coils mate with the separated, proximal end member coils around the mandril wire 52 for releasably attaching the handle 46 from the filler member 18. A length of platinum wire may be wrapped for a length of 2 to 3 mm between the most proximal of the separated coils of the handle. The platinum wire wrap may be attached to the stainless steel coils by soldering or any other well-known method. The proximal end of the stainless steel coil forming the handle may be soldered to an appropriate length of 23 gauge cannula. The length of cannula may include two right angle bends with a proximally extending straight portion approximately 2 cm long.

Where the filler member 18 is a coil, the appropriate lengths of the stainless steel coil of the handle 46 and the filler member 18 may be varied depending on the size of the expandable occlusion bag 14. For example, a 3 mm wide bag may be used with a 10.5 cm length of filler member coil 18 and an 83.5 cm length of handle coil 46; a 5 mm wide occlusion bag 14 may be used with an 18 cm length of filler member coil 18 and a 91 cm length of handle coil 46; a 7 mm wide bag 14 may be used with a 31 cm length of filler member coil 18 and a 104 cm length of handle coil 46; and a 9 mm wide bag 14 may be used with a 53 cm length of filler member coil 18 and a 126 cm length of handle coil 46.

FIG. 6A depicts a representative longitudinal view of a percutaneous release mechanism where the filler member 18 is in the form of submucosal material. Of course, other bioremodelable materials may be used instead. Although the filler member 18 in FIG. 6A is depicted in the form of threaded submucosal material, other bioremodelable materials or forms of submucosal may be used as described above. In FIG. 6A, the positioning catheter 34 enclosed within the pusher catheter 44, encloses an inner member 60 for pushing the submucosal material into the occlusion bag 14 in which it is retained. Inner member 60 is depicted in FIG. 6A as having a distal segment 62 having a smaller cross-sectional area than the proximal segment 64 of the inner member 60 and a ball 66 at the distal end of the inner member 60. However, the inner member may be in the form of a simple rod or any other shape suitable for pushing submucosal materials or other bioremodelable and/or filler member materials.

Various filler member retainment means may be employed to retain the filler member 18 in the occlusion bag 14, prevent its release after delivery of the occlusion bag to an occlusion site, and/or seal the expandable occlusion bag 14. FIGS. 6A and 6B depict an embodiment in which a check flow flap 62 is incorporated on the inside of the occluding bag to restrict outward movement of filler material. The check flow flap 62 is biased toward sealing the end of the occlusion bag 14 upon retraction of inner member 60 therefrom.

FIG. 7A-7C depict a means for sealing the end of the occlusion bag 14, which incorporates a plug or wedge 68 behind the filler member 18, depicted here in the form of submucosal material. Of course, other bioremodelable materials may be used instead. A representative plug or wedge may be in the form of e.g., a spongy PVA plug, although other types of plugs or wedges used in the art for sealing openings may be used also. Additional filler member retainment means may include the use of a spring biased collar 30 to aid in sealing the end of the occlusion bag 14 upon retraction of the positioning catheter 34 or the inner member 60.

The pusher type connection release mechanism depicted in FIG. 6-7 may be adapted to any type of filler member 18 in accordance with the present invention, and may be used where the filler member is in the form of coils, filler wires and/or bioremodelable or submucosal materials. Representative teachings for release of such materials are set forth in U.S. Pat. Nos. 5,417,708 and 5,562,698.

It is to be understood that the above-described vascular occlusion devices and assemblies are merely representative embodiments illustrating the principles of this invention and that other variations in the devices, assemblies, or methods, may be devised by those skilled in the art without departing from the spirit and scope of this invention.

What is claimed:

1. An occlusion device comprising:
an expandable occlusion bag having an internal cavity and an opening in communication thereto; and
a filler member,
where at least one of the expandable occlusion bag and the filler member comprise bioremodelable material and where the filler member is adapted for insertion in the internal cavity through the opening of the expandable occlusion bag to substantially fill the internal cavity of the bag, thereby expanding the shape of the bag to facilitate occlusion in a body vessel.

2. The occlusion device of claim 1, where the bioremodelable material comprises a natural source of tissue material.

3. The occlusion device of claim 1, where the expandable occlusion bag comprises bioremodelable material.

4. The occlusion device of claim 1, where the expandable occlusion bag comprises nylon.

5. The occlusion device of claim 2, where the filler member comprises a coil or a forming wire.

6. The occlusion device of claim 1, where the filler member comprises bioremodelable material.

7. The occlusion device of claim 1, where each of the expandable occlusion bag and the filler member comprises bioremodelable material.

8. The occlusion device of claim 1, where at least one of the expandable occlusion bag and the filler member comprises a plurality of submucosal layers laminated together.

9. The occlusion device of claim 1, further comprising marker materials rendering the device radiopaque or MRI compatible.

10. The occlusion device of claim 1, where the expandable occlusion bag comprises a filler member retainment means.

11. The occlusion device of claim 10, where the filler member retainment means includes the use of a check flow flap to seal the opening of the expandable occlusion bag.

12. The occlusion device of claim 10, where the filler member retainment means includes the use of a plug to seal the opening of the expandable occlusion bag.

13. An occlusion assembly comprising:
an expandable occlusion bag having an internal cavity and an opening in communication thereto;
a positioning catheter having a distal end joined to the external opening of the expandable occlusion bag, said catheter adapted for positioning the occlusion bag in a vessel of a patient;
a pusher catheter coaxially associated with the positioning catheter; and
a filler member,
where at least one of the expandable occlusion bag and the filler member comprise bioremodelable material and where the filler member is adapted for insertion in the internal cavity through the opening of the expandable occlusion bag to substantially fill the internal cavity of the bag, thereby expanding the shape of the bag to facilitate occlusion in a body vessel.

14. The occlusion assembly of claim 13, further comprising a handle positioned in the positioning catheter, where actuation of the handle facilitates transfer of the filler member into the expandable bag.

15. The occlusion assembly of claim 13, further comprising an introducer sheath positioned coaxially over the positioning catheter and the pusher catheter.

16. The occlusion assembly of claim 13, comprising a filler member retainment means for retaining the filler member in the expandable occlusion bag.

17. A method of occluding a vessel in a patient comprising:
a. providing an occlusion device in a patient, said occlusion device comprising:
i. an expandable occlusion bag having an internal cavity and an opening in communication thereto; and
ii. a filler member, where at least one of the expandable occlusion bag and the filler member comprise bioremodelable material and where the filler member is adapted for insertion in the internal cavity through the opening of the expandable occlusion bag to substantially fill the internal cavity of the bag, thereby expanding the shape of the bag to facilitate occlusion in a body vessel;
b. positioning the expandable occlusion bag in a vessel site of the patient;
c. transferring the filler member into the internal cavity of the occlusion bag;
d. expanding the occlusion bag with the filler member so that the occlusion bag expands, thereby occluding the vessel.

18. The method of claim 17, where the step of transferring the filler member comprises a screw type or pusher type connection release mechanism.

19. The method of claim 17, further comprising a filler member retainment means for retaining the filler member in the expandable occlusion bag.

20. The method of claim 17, further comprising the step of allowing the occlusion bag and filler member to undergo remodeling where the bioremodelable material is degraded and replaced by endogenous tissue.
